(19) United States
(12) Patent Application Publication
(10) Pub. No.: US 2024/0277884 A1
(43) Pub. Date: Aug. 22, 2024

(54) High efficiency generation of a vapor-containing aerosol mist without added heat

(71) Applicant: Teknologian tutkimuskeskus VTT Oy, Espoo (FI)

(72) Inventor: Jani Hakala, Espoo (FI)

(21) Appl. No.: 18/571,268

(22) PCT Filed: Jun. 23, 2022
(86) PCT No.: PCT/FI2022/050461

§ 371 Date: Dec. 18, 2023

(30) Foreign Application Priority Data
Jun. 23, 2021 (FI) 20215739

(51) Int. Cl. A61L 9/14; A61L 101/02; B05B 7/00; B05B 7/26; B05B 17/06

(52) U.S. Cl. CPC A61L 9/145; A61L 2101/02; A61L 2209/132; A61L 2209/134; B05B 7/0012; B05B 7/262; B05B 17/0615

(57) ABSTRACT

The present invention concerns a device and a method for high efficiency generation and discharge of an at least partially evaporated aerosol mist from a sterilizing solution with no added heat. Particularly, the device and method are suitable for generation of hydrogen peroxide vapor.

### BACKGROUND OF THE INVENTION

### Field of the Invention

The present invention concerns a device and a method for the generation and discharge of a vapor-containing aerosol mist produced from a sterilizing solution at ambient temperature. Particularly, the device and method are suitable for generation of hydrogen peroxide vapor.

### Description of Related Art

When cleaning and disinfecting areas that require high purity, such as areas in hospitals or food and beverage industry, the focus is usually on the visible surfaces. However, many pathogens are airborne, and may end up on hard to reach surfaces and in the air ducts. Therefore, wiping the visible surfaces with a disinfectant is not sufficient. Sterilizing or disinfecting vapors or sprays have thus been developed. For example U.S. Pat. No. 2004005240 A1 describes the generation of a sterilizing mist, where the sterilizing solution is discharged in droplet form.

Vapors are typically more gentle than liquids, and especially oxidizing agents may cause corrosion or bleaching in the liquid phase. Vapors are also more mobile, and will reach underside surfaces more efficiently than liquid droplets.

CN 111658803 describes a cavity for sterilizing objects placed therein using a rapidly evaporated hydrogen peroxide spray that is discharged directly onto the object. However, although small droplets will evaporate easily, the larger droplets will not, and they may also act as a seed droplet for the vapors to re-condensate on, whereby the vapor generation is not as efficient, and the large droplets exiting the generator may deposit on surfaces and cause risk for corrosion or bleaching.

In vapor generation, most techniques involve heating to evaporate liquid solutions of sterilizing agent. However, heating may cause decomposition of the sterilizing agent. This is known to take place e.g. with hydrogen peroxide.

Such heat-induced vapor generation for disinfecting purposes has been described e.g. in DE 102008050947, wherein droplets of hydrogen peroxide are fed into a heated container from the top of the container using a pump, and are vaporized by a stream of pre-heated air that is blown against the droplets, the heat causing the evaporation of the entire size-spectrum of droplets.

Consequently, there is still a need for new techniques for generating vapor from sterilizing solutions, and for discharging said vapor into a space to be sterilized, disinfected or decontaminated.

### SUMMARY OF THE INVENTION

The invention is defined by the features of the independent claims. Some specific embodiments are defined in the dependent claims.

According to a first aspect of the present invention, there is provided a device and a method for the generation and discharge of an at least partially evaporated aerosol mist of a sterilizing solution.

According to a second aspect of the present invention, there is provided such a device and method for the generation and discharge of a vapor-containing mist at ambient temperature.

To achieve efficient evaporation of the sterilizing solution, the device includes means for separating large droplets of the sterilizing solution from a formed mist, thus allowing only a fine mist containing small droplets to continue out from the device.

Similarly, the invention relates to a method for such vapour generation and discharge, including said separation of the largest droplets of sterilizing solution from the mist.

Several advantages are achieved using the present invention. Among others, vapour can be generated without added heat, due to the separation of the largest droplets of the sterilizing solution.

Providing a mist of only the remaining small droplets means that evaporation will be more efficient and no seed droplets will be present to cause re-condensation. A further advantage of the invention is that vapour can be generated in a more gentle manner, as compared to conventional techniques, without the risk for decomposition of the sterilizing agent. Due to the lack of heating and the lack of extensive vaporization control, the device can also be kept more simple than bulky commercial units.

If using a hydrogen peroxide-based sterilizing solution, there is the additional advantage of using a sterilizing mist that leaves no harmful chemical residue.

### BRIEF DESCRIPTION OF THE DRAWINGS

FIG. 1 illustrates the structure of the device in accordance with at least some embodiments of the present invention, the dotted arrows representing streams of gas, solution or mist;

FIGS. 2A and B illustrate the structure of the device in accordance with some preferred embodiments of the present invention, with different atomizers.

FIG. 3 illustrates the structure of the device in accordance with other embodiments of the invention, with a preferred solution feed system.

FIGS. 4A-C illustrate the structure of the device in accordance with some further embodiments of the invention, with alternative droplet separation units.

FIG. 5 illustrates the structure of the device showing an optional gas distributor.

### EMBODIMENTS OF THE INVENTION

### Definitions

In the present context, the term “aerosol” is intended to encompass a suspension of fine liquid droplets in a gas. While suspended into a streaming gas, it forms a mist that can be sprayed e.g. into an area to be decontaminated. Further, the small size of the fine liquid droplets of the mist allow for evaporation of at least a fraction of the droplets.

The term “fine liquid droplets” of the mist is, in turn, intended to define droplets that have a sufficiently small inertia that allows them to be carried in the trajectory of a gas stream, even when the gas stream changes its direction. Typically, such small droplets of the mist have a diameter of <10 μm, preferably <5 μm, giving them a sharp surface curvature that facilitates evaporation.

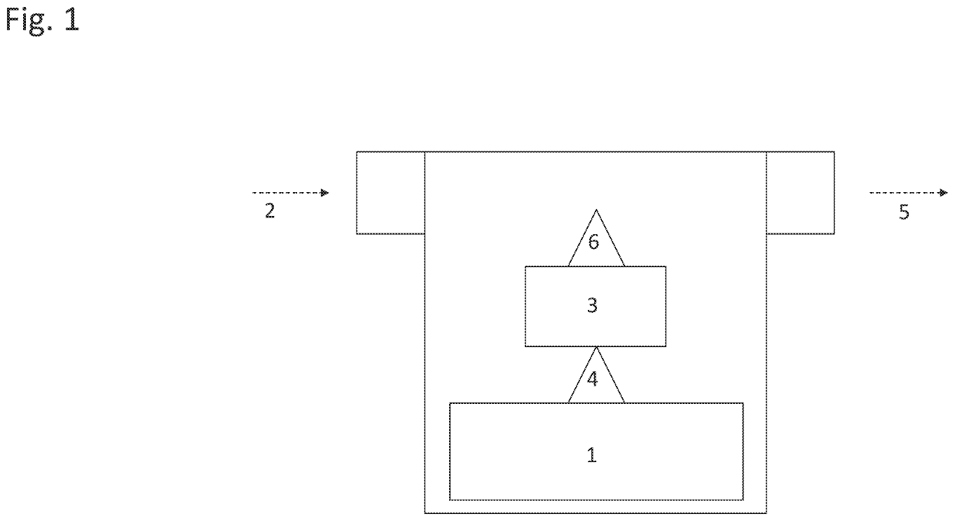

The present invention thus relates to a device for the generation and discharge of an at least partially evaporated aerosol mist from a sterilizing solution (see FIG. 1), typically operated at ambient temperature, the device being formed of a container comprising
a reservoir 1 for holding the sterilizing solution,
a gas inlet 2, for carrying a stream of gas into the container,
an atomizing unit 3 for forming an aerosol mist from the sterilizing solution,
an optional feed system 4 for supplying sterilizing solution to the atomizing unit 3, and
a mist outlet 5, for leading a formed vapor-containing mist out of the container.

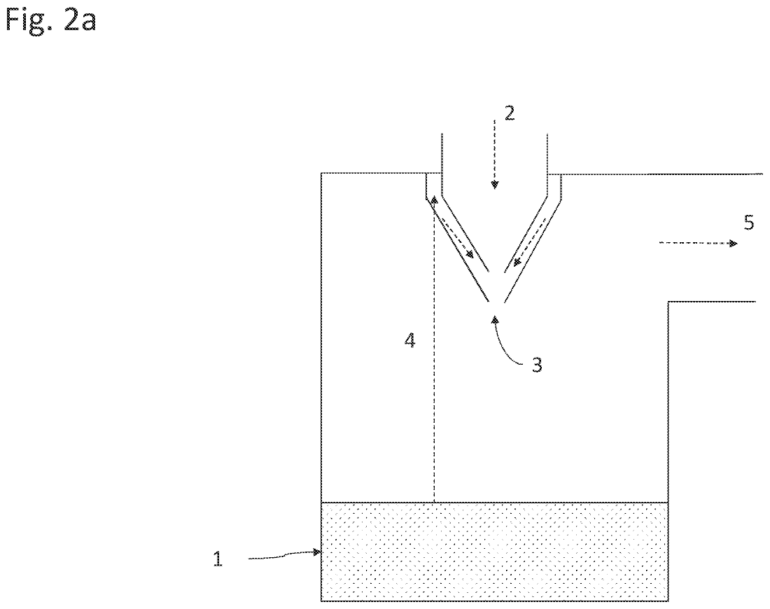

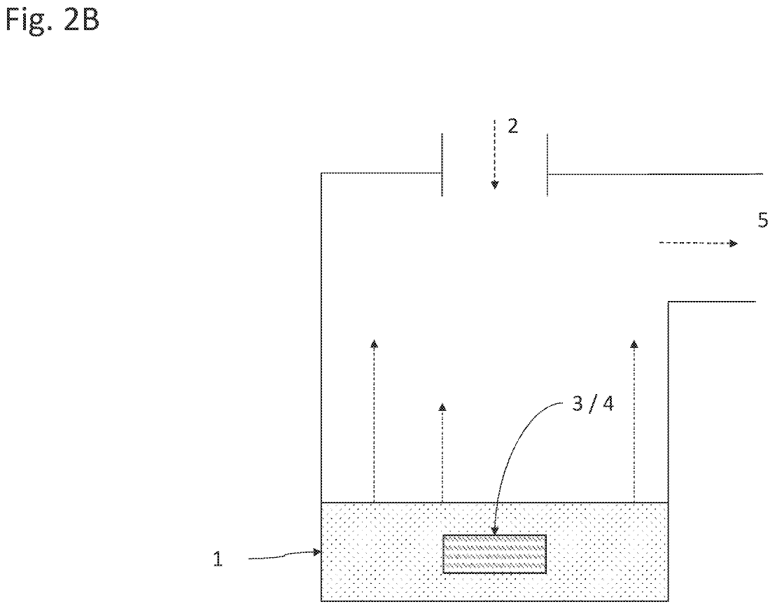

As shown in FIGS. 2A-B, the atomizing unit 3 typically comprises one or more of the following:
one or more misting nozzles (see FIG. 2A), arranged either to mix the sterilizing solution with a created high velocity jet of the stream of gas, or arranged to receive the sterilizing solution that is pumped through the nozzle at high pressure,
one or more vibrating orifices, or
one or more ultrasonic misters (FIG. 2B),
or more than one of these atomizers 3.

In one embodiment of the invention, the atomizing unit 3 is a misting nozzle having an outlet with a width of 0.01-10 mm, preferably 0.2-3.0 mm, more preferably 0.5-1.5 mm.

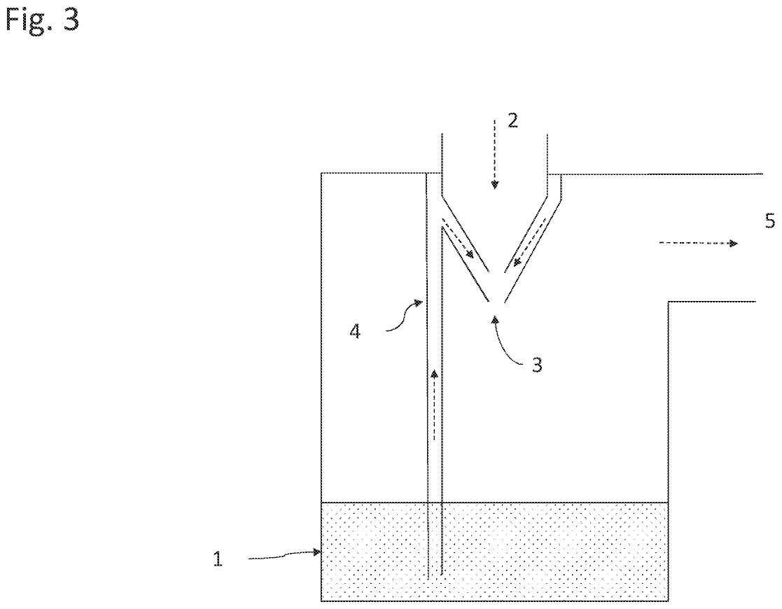

As indicated above, the device may optionally comprise a feed system 4 for supplying sterilizing solution from the reservoir 1 to the atomizing unit 3. This feed system 4 may be in the form of
one or more vacuum ejectors (see FIG. 3), arranged to transfer sterilizing solution from the reservoir 1 via a liquid channel to the atomizing unit 3 by means of Venturi effect,
one or more liquid pumps, arranged to pump the sterilizing solution from the reservoir 1 via a liquid channel to the atomizing unit 3, or
a liquid channel arranged to feed the sterilizing solution from the reservoir 1, placed at an elevated position, to the atomizing unit 3 by gravitational forces.

However, when the device comprises an atomizing unit 3 that is submerged into the reservoir 1 containing the sterilizing solution, such as an ultrasonic mister, no separate feed system 4 is needed.

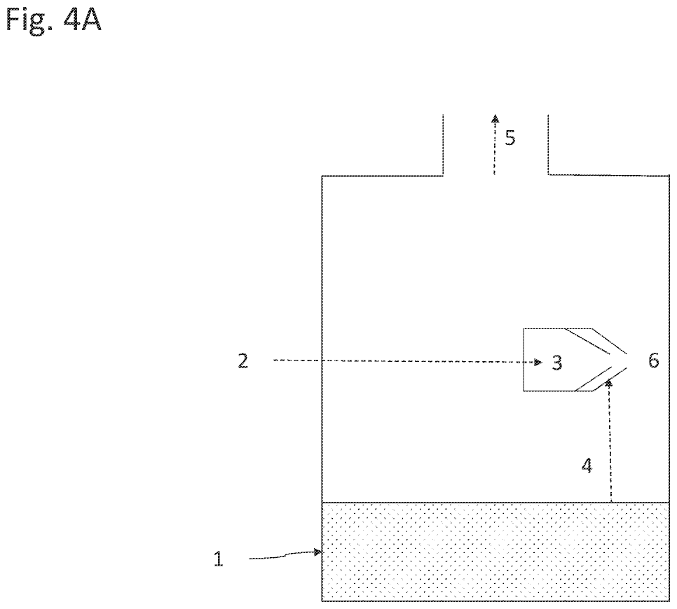

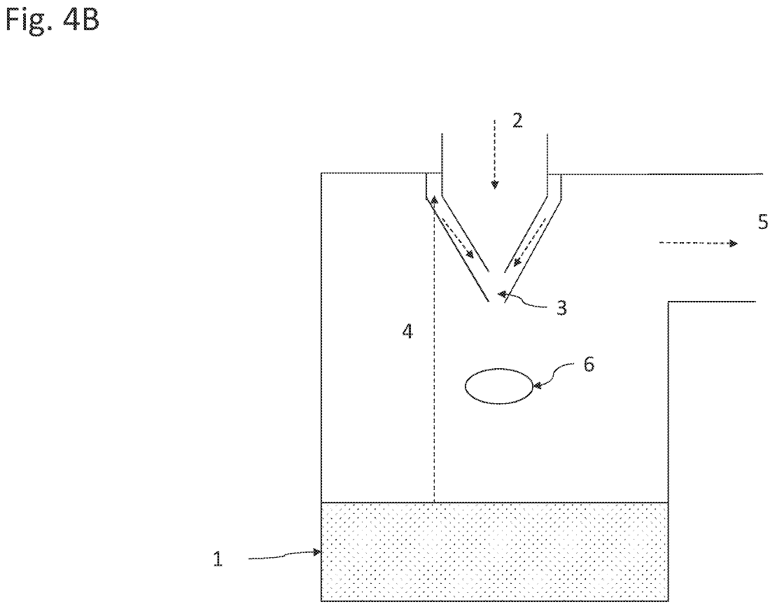

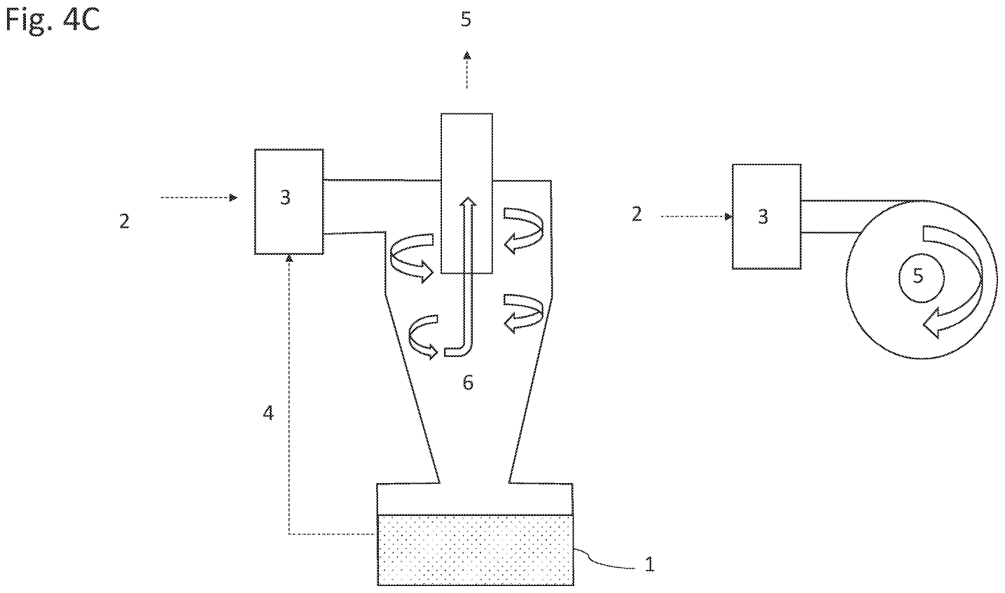

Further to the above, the device comprises an inertial separation unit 6 (see FIGS. 4A-C) arranged to cause collision of a fraction of the droplets of the aerosol against a surface, such as the surface of a wall of the container (see FIG. 4A), thus separating the collided droplets from the aerosol.

The fraction of the droplets that are separated from the mist are typically the largest droplets, which are too heavy to change their direction when facing a surface, thus leaving only the easily evaporated small droplets in the mist.

In an embodiment of the invention, the inertial separation unit 6 is in the form of an impactor, such as an impactor with an impaction target (see FIG. 4B), or a cyclone separator (see FIG. 4C), preferably in the form of an impaction target positioned in the trajectory of the stream of gas passing the atomizing unit 3, thus causing the collision of large droplets of the solution against said target, causing either their atomization or their return to the reservoir 1.

Particularly, the inertial separation unit 6 is in the form of an impaction target, placed at a distance from the outlet of the atomizing unit (3) that is 0.1-10 times the width of the outlet, preferably at a distance of 1-4 times the width of the outlet, more preferably at a distance of 1.5-2.5 times the width of the outlet.

To facilitate the separation of droplets, the atomizing unit 3 may further be positioned at or preferably below the level of the mist outlet 5 in the vertical, for preventing relatively large droplets from being discharged through the mist outlet 5.

Further, in a preferred embodiment of the invention, the gas inlet 2 and the mist outlet 5 may be positioned at an angle to each other, to cause the mist mixed in the stream of gas to change its direction within the container, thus causing large droplets to hit a surface of the container, while small droplets change their direction, and are carried with the stream of gas to the mist outlet 5. Thus, it is preferred to:
position the gas inlet 2 to create a trajectory for the gas stream through the gas inlet 2 having a first orientation,
position the mist outlet 5 to create a trajectory for the gas stream through the mist outlet 5 having a second orientation, and
to limit the angle between the first orientation in respect to the second orientation to an angle between 60 and 120°,
   thus leading relatively large droplets towards the walls of the container preferably to be returned to the reservoir 1 instead of being discharged through the mist outlet 5.

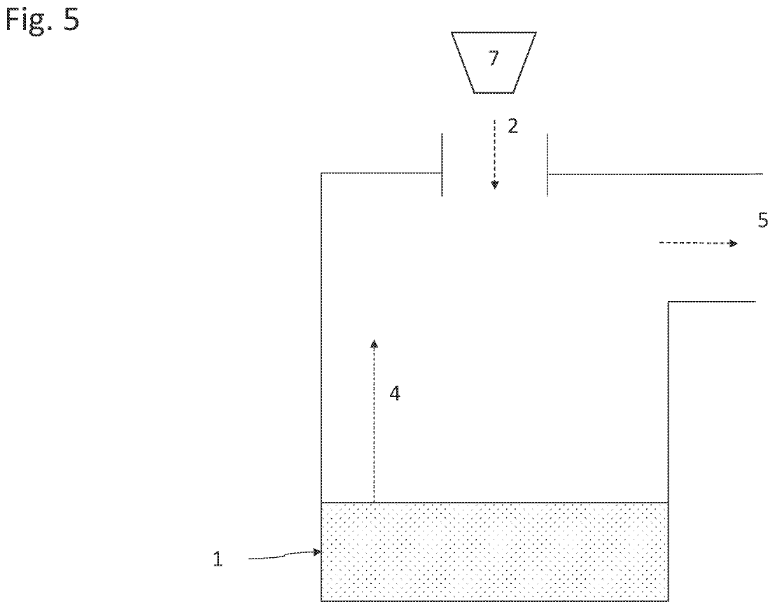

As shown in FIG. 5, the gas inlet 2 can further be placed in fluid communication with a gas distributor 7, preferably being in the form of a gas pressurizer, a fan or a blower, more preferably in the form of a gas pump, a compressor or a container holding a pressurized gas, most suitably being in the form of a compressed or liquidized gas cylinder.

The preferred means for providing pressurized gas are useful when a stream of high velocity gas is used for the vaporization of the droplets.

In an alternative embodiment, when using pressurized gas, also a separate inlet for flushing gas can be included in the device. This separate flushing gas will create a further gas flow into the container, thus facilitating the discharge of the generated mist and enhancing the diffusion and evaporation of the droplets in the gas stream discharged through the mist outlet 5.

Since many sterilizing solutions consist of highly reactive agents, such as strong oxidants, the device is typically made of durable material, not interfering with the sterilizing solution. Preferred materials are plastic, glass, or metal, more preferably being plastic or glass. For example, when using a sterilizing solution containing hydrogen peroxide, it is preferred to avoid metals.

The device of the invention can be used for instance in the method of the invention, also intended for the generation and discharge of an at least partially evaporated aerosol mist from a sterilizing solution, at ambient temperature. Said method comprises the steps of
carrying a stream of gas into a container holding a sterilizing solution,
leading the stream of gas through the container, into an atomizing unit 3,
providing sterilizing solution at the atomizing unit 3, optionally using a separate step of feeding the sterilizing solution to the atomizing unit 3,
atomizing the sterilizing solution in the atomizing unit 3, thus forming an aerosol mist containing at least partially evaporated droplets of the solution, and
leading the formed aerosol mist with the gas stream through a mist outlet 5 out of the container, where the possible remaining fine mist droplets continue the evaporation process at fast rate.

The gas used in the method can be any gas, preferably an inert gas, and more preferably a gas selected from nitrogen, carbon dioxide, oxygen, argon or air.

The aerosol mist that is carried to the mist outlet 5 with the help of the gas stream is formed by atomizing the liquid solution into fine droplets, preferably
by driving the liquid solution through one or more misting nozzles, where it is mixed with a created high velocity jet of the stream of gas,
by pumping the liquid solution at high pressure through one or more misting nozzles,
by driving the liquid solution through one or more vibrating orifices, or
by using one or more ultrasonic misters,
or by using two or more of these alternatives simultaneously.

As stated above, the sterilizing solution may optionally be fed to the atomizing unit 3 in a separate feeding step, although this feeding can be dispensed of when the atomizing unit 3 is immersed into the sterilizing solution. If used, the separate feeding of the sterilizing solution can be carried out by
using one or more vacuum ejectors, which transfer sterilizing solution by means of underpressure created by Venturi effect,
pumping the sterilizing solution using one or more liquid pumps, or
using gravitational forces to feed the sterilizing solution.

The method of the invention is characterized by separating a fraction of the droplets of the sterilizing solution from the gas stream via collision, preferably by colliding the droplets against a surface in the container, such as the surface of a wall of the container, or alternatively the surface of an impaction target, thus causing separation of the collided droplets from the gas stream.

Typically, the largest droplets will collide against the surface, and will either be separated from the gas stream or atomized into smaller droplets, whereby the aerosol mist that is carried with the gas stream to the mist outlet 5 includes only relatively small droplets, which are easily evaporated at ambient temperature.

The liquid from the largest droplets separated from the gas stream is preferably returned to the solution in the container.

In a preferred embodiment, the droplet separation takes place using inertial separation, more preferably by using an impactor or a cyclone separator, most suitably by using an impaction target positioned in the trajectory of a high velocity stream of gas, typically positioned so that large droplets of the solution captured by the gas stream hit the target and are either atomized into smaller droplets or returned to the solution reservoir 1, where the solution can be reused.

The separation of a fraction of droplets can also be facilitated by positioning the mist outlet 5 in the container at a position that forces the stream of gas to change its direction within the container, thus causing inertial separation of large droplets from the stream containing the mist.

For example, large droplets can thus be separated from the mist by positioning the gas inlet 2 and the mist outlet 5 at an angle in respect to each other, whereby the trajectory of the gas stream flowing from the gas inlet 2 to the mist outlet 5 is forced to make a turn. A suitable angle is between 60 and 120°. This will lead relatively large droplets towards the walls of the container instead of being discharged through the mist outlet 5.

Using one or more of these techniques for separating a fraction of droplets from the gas stream, the diameter of the droplets in the formed aerosol mist is typically limited to a droplet diameter of <10 μm, preferably <5 μm, via the separation of a fraction of the droplets including the largest droplets of the mist.

Due to the small size of the droplets remaining in the mist, the evaporation is spontaneous, thus resulting in a vapour-containing mist. The evaporation is aided by the sharp surface curvature of such small droplets.

As stated above, the gas used in the method can be any suitable gas. Particularly, the gas can be selected from nitrogen, carbon dioxide, oxygen, argon or air, or another suitable gas, preferably being air.

In those embodiments requiring a high-velocity gas stream, the gas is preferably supplied at high pressure, such as a pressure of >1 bar, more preferably at a pressure of 2-10 bar. One alternative is also to use a separate flushing gas to facilitate carrying the formed mist out of the container, which can be selected from the above mentioned list of gases.

The sterilizing solution can be selected from any solution containing one or more sterilizing agents, which are volatile enough to be evaporated from small droplets at ambient temperature. It can be used as a 100% solution of the sterilizing agent(s), or it can be diluted with water or other solvent, depending on the selected agents. A preferred sterilizing agent is an aqueous solution of hydrogen peroxide, which may be mixed with additives, such as ammonia or acetic acid.

Also the gas can be mixed with additives, such as ozone, ethylene oxide, ammonia, chlorine, chlorine dioxide or nitrogen dioxide, to further enhance the sterilization process, and provide a synergistic effect.

In a preferred embodiment, the sterilizing solution is a solution containing hydrogen peroxide in water, preferably in a hydrogen peroxide concentration of 1-100 vol-%, more preferably in a concentration of 30-75 vol-%, even more preferably in a concentration of 30-65 vol-%, most suitably in a concentration of 35-50 vol-%, the method thus forming hydrogen peroxide vapor.

One alternative is also to add acetic acid to the preferred hydrogen peroxide solution, particularly in a concentration of ≤10 mass %, preferably ≤7 mass %, and most suitably in a concentration of about 5 mass %.

The above described device and method are suitable for use e.g. in disinfecting or decontaminating air and surfaces in a target volume, preferably in a closed space, such as in a room or an air duct. In such a use, a small concentration of the mist of the invention is sufficient, e.g. a concentration of 150-1000 ppm, and allowing the mist to react for >5 min, preferably >1 h.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In this description, numerous specific details are provided to provide a thorough understanding of embodiments of the invention. One skilled in the relevant art will recognize, however, that the invention can be practiced without one or more of the specific details.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

### INDUSTRIAL APPLICABILITY

The present device and method can be used for heatless high efficiency vapor generation, and generally for replacement of conventional devices for generation and discharge of vapor-containing mists of various sterilizing solutions.

Reference Signs List

| | |
|---|---|
| 1 | reservoir |
| 2 | gas inlet |
| 3 | atomizing unit |
| 4 | optional feed system |
| 5 | mist outlet |
| 6 | inertial separation unit |

and preferably

| | |
|---|---|
| 7 | gas distributor |

### CITATION LIST

### Patent Literature

CN 111658803
DE 102008050947
U.S. Pat. No. 2004005240 A1

1. A device for the generation and discharge of an at least partially evaporated aerosol mist from a sterilizing solution, the device being formed of a container comprising:
a reservoir for holding the sterilizing solution,
a gas inlet for carrying a stream of gas into the container,
an atomizing unit for forming an aerosol mist from the sterilizing solution,
an optional feed system for supplying sterilizing solution to the atomizing unit, and
a mist outlet for leading a formed vapor-containing mist out of the container,
wherein:
the device further comprises an inertial separation unit arranged to cause collision of a fraction of the droplets of the aerosol against a surface of the container, thus separating the collided droplets from the aerosol, the inertial separation unit being in the form of an impaction target, placed in the trajectory of the stream of gas passing the atomizing unit at a distance from the outlet of the atomizing unit that is 1-4 times the width of the outlet.

2. The device of claim 1, wherein the atomizing unit comprises one or more of the following:
one or more misting nozzles, arranged either to mix the sterilizing solution with a created high velocity jet of the stream of gas, or arranged to receive the sterilizing solution that is pumped through the nozzle at high pressure,
one or more vibrating orifices, or
one or more ultrasonic misters.

3. The device of claim 1, wherein the atomizing unit is a misting nozzle having an outlet with a width of 0.01-10 mm.

4. The device of claim 1, wherein the impaction target is placed at a distance from the outlet of the atomizing unit that is 1.5-2.5 times the width of the outlet.

5. The device of claim 1, wherein the device further comprises a feed system for supplying sterilizing solution to the atomizing unit is in the form of:
one or more vacuum ejectors, arranged to transfer sterilizing solution from the reservoir via a liquid channel to the atomizing unit by means of Venturi effect,
one or more liquid pumps, arranged to pump the sterilizing solution from the reservoir via a liquid channel to the atomizing unit, or
a liquid channel arranged to feed the sterilizing solution from the reservoir, placed at an elevated position, to the atomizing unit by gravitational forces.

6. The device of claim 1, wherein the device further comprises an atomizing unit submerged into the reservoir containing the sterilizing solution, whereby no separate feed system is needed.

7. The device of claim 1, wherein the device is made of durable material selected from the group consisting of plastic, glass, and metal.

8. The device of claim 1, wherein the gas inlet is in fluid communication with a gas distributor, preferably being in the form of a gas pressurizer, a fan or a blower, more preferably in the form of a gas pump, a compressor or a container holding a pressurized gas, most suitably being in the form of a compressed gas bottle.

9. A method for the generation and discharge of an at least partially evaporated aerosol mist from a sterilizing solution at ambient temperature, comprising the steps of:
carrying a stream of gas into a container holding a sterilizing solution,
leading the stream of gas through the container, to an atomizing unit,
providing sterilizing solution at the atomizing unit,
atomizing the sterilizing solution in the atomizing unit, thus forming an aerosol mist containing at least partially evaporated droplets, and
leading the formed aerosol mist with the gas stream through a mist outlet out of the container, and
separating a fraction of droplets of sterilizing solution from the gas stream via collision against an impaction target, placed in the trajectory of the stream of gas passing the atomizing unit at a distance from the outlet of the atomizing unit that is 1-4 times the width of the outlet, thus separating the collided droplets from the gas stream carried to the mist outlet.

10. The method of claim 9, wherein the aerosol mist is formed by atomizing the liquid solution:
by driving the liquid solution through one or more misting nozzles, where it is mixed with a created high velocity jet of the stream of gas,
by pumping the liquid solution at high pressure through one or more misting nozzles,
by driving the liquid solution through one or more vibrating orifices, or
by using a separate ultrasonic mister,
or by using two or more of these alternatives simultaneously.

11. The method of claim 9, wherein the gas is nitrogen, oxygen, carbon dioxide, argon or air.

12. The method of claim 11, wherein the gas further comprises ozone, ethylene oxide, ammonia, chlorine, chlorine dioxide or nitrogen dioxide as an additive.

13. The method of claim 9, wherein the sterilizing solution is fed to the atomizing unit in a separate feeding step by:
using one or more vacuum ejectors, which transfer sterilizing solution by Venturi effect,
pumping the sterilizing solution using one or more liquid pumps, or
using gravitational forces to feed the sterilizing solution.

14. The method of claim 9, wherein a fraction of droplets of sterilizing solution is separated from the gas stream by colliding the fraction of droplets against either a surface of the container or a surface of the impaction target, the collided droplets being the largest droplets of the solution, thus causing either the atomization of these collided droplets or their return to the solution in the container.

15. The method of claim 9, wherein the diameter of the droplets in the formed aerosol mist is limited to a droplet diameter of <10 μm via the separation of a fraction of the droplets including the largest droplets of the mist.

16. The method of claim 9, wherein the sterilizing solution is a solution containing hydrogen peroxide in water in a concentration of 1-100 vol-%, the method thus forming hydrogen peroxide vapor.

17. (canceled)
